(19) United States
(12) Patent Application Publication
(10) Pub. No.: US 2004/0167596 A1
(43) Pub. Date: Aug. 26, 2004

(54) Method and apparatus for orientation of an implantable device

(76) Inventor: Jacob Richter, Ramat Hasharon (IL)

Correspondence Address: MORGAN & FINNEGAN, L.L.P., 345 Park Avenue, New York, NY, 10154-0053, US

(21) Appl. No.: 10/372,699

(22) Filed: Feb. 24, 2003

(51) Int. Cl. A61F 2/06; A61B 5/00

(52) U.S. Cl. 623/1.1; 600/341

(57) ABSTRACT

A method and apparatus for orienting an implant such as a sensor or other device in a bodily lumen with an external source such as a transmitter or transducer. The apparatus includes a fastener having at least one support leg and a fixture for holding the implant. The implant, fixture, or both may move to achieve alignment of the implant with the external source Mechanical alignment may be used by moving a catheter such as one used to place the implant and fastener in the lumen to align the sensor. A second type of alignment based on gravity may be used so that when a weighed implant is used a patient while lying in certain positions would force the implant into alignment with the source. A third type of alignment uses magnetic force having a magnet on the implant, source or both for aligning the implant relative to the source.

### FIELD OF INVENTION

The method and device disclosed herein generally relates to the field of medical implants and more particularly to a method and device for orientating a device, including but not limited to, a sensor in a bodily lumen such that the sensor or other implantable device is aligned with an external transmitter or transducer that allows, among other features, measurements to be taken within the bodily lumen.

### BACKGROUND

In the medical arts, it is becoming more desirable to use small implantable biological devices and sensors or “biodevices” and “biosensors” to affect or measure the responses of organs in a patient's body. For example, the continuous measurement of blood flow in vessels, particularly in postoperative cases, is desirable for the evaluation of vascular reconstructive procedures, and in some instances to change a vessel's physiological parameters.

An example of such medical procedure is angioplasty or percutaneous transluminal coronary angioplasty (PTCA). Angioplasty is a medical technique for reopening blocked arteries in the heart without major surgery. Angioplasty is also used in other parts of the body, typically to treat peripheral artery disease. The angioplasty procedure is performed while the patient is awake, under local anesthesia. The surgeon typically makes an incision in the groin or arm and then carefully inserts contrast dye into patient's blood stream. The dye is used to show the arteries on an x-ray or other imaging apparatus. A catheter with a balloon attached is guided to the blocked artery. The balloon catheter is inserted inside the narrow part of the blocked blood vessel and inflated. Fatty deposits and plaque covering the walls of the artery are compressed into the wall of the artery increasing the diameter of the previously blocked artery. Stents or small metal tubes are often inserted and expanded in the artery to provide support to the newly opened vessel.

It frequently occurs, however, that the vessel narrows again after the procedure, either mechanically or by cell proliferation. Thus, in many cases, an angioplasty procedure must be repeated. It would be desirable to monitor the flow of blood inside the stent so as to determine whether and when significant narrowing has occurred.

In other cases, it may be desirable to monitor of blood flow parameters in the pulmonary artery to have early warning for Congestive Heart Failure (CHF), or monitor blood pressure outside a repair device in an abdominal aortic aneurysm (AAA) to sense the degree of sealing of the aneurysmal sack.

Devices, for example, that can be placed in the body and activated or monitored from the outside, typically require alignment with an external transmitter or transducer so that an accurate measurement can be taken with optimal signal to noise ratio. A number of attempts have been made in the past to develop and use in vivo devices or sensors for the continuous monitoring of various conditions. However, such sensors have been plagued by a number of deficiencies, most notably inaccuracies and the inability of the sensors to remain in place over a long period of time.

In addition, alignment of these devices and sensors relative to the transmitter or transducer is critical to their accurate operation. The range for proper alignment or the angle tolerance of the device or sensor relative to the transmitter or transducer may be quite narrow. Therefore, orientation is a critical component for successful use in vivo.

Thus, a need exists for a device and method of orientating a device or sensor relative to an external transmitter or transducer such that the component has the ability to remain in place over an extended period of time.

### SUMMARY OF THE INVENTION

The present invention avoids many of the disadvantages of previous implant positioning apparatus. In accordance with one embodiment, an orientation device and method of use for aligning an implant, such as a sensor or other device, in a bodily lumen relative to a target exterior to a patient's body includes a fastener, a fixture, and a sensor or other device. The target that is exterior to the patient can be a transmitter, or transducer or the like. The sensor or device is connected to the fastener, also known as a fixation structure, by a joint. The joint allows the sensor or device to move relative to the target.

Orientation adjustments are achieved either manually or automatically. In one embodiment, a catheter is used to mechanically align the sensor or device. In another embodiment, the implant is automatically oriented using gravitational forces by positioning the patient. In this embodiment, a bias weight can be incorporated into the implant to allow the orientation to react to changes in position of the patient. In yet another embodiment, a magnet is attached to the implant, and alignment is made by application of a magnetic field.

These, and other aspects of the apparatus and method of use of the orientation device, are described in the following brief and detailed description of the drawings.

### BRIEF DESCRIPTION OF THE DRAWINGS

In the accompanying drawings which form a part of the specification and are to be read in conjunction therewith and in which like reference numerals are used to indicate like parts in the various views. The drawings are not drawn to scale and in fact magnify the sensor or device in the vessel, in order to more particularly illustrate their details.

FIG. 1 is a cross-sectional view of an orientation device in a bodily lumen for aligning a device, such as for example, a sensor with an external transducer;

FIG. 2 is a cross-sectional view of the orientation device in FIG. 1 using one sensor and multiple support legs;

FIG. 3 is an enlarged cross-sectional view of the orientation device in FIG. 1 showing rotation of a fixture;

FIG. 4 illustrates an enlarged cross-sectional side view of one embodiment of a rotational device used to rotate and lock in position a sensor, a fixture or both the sensor and the fixture;

FIG. 5A shows an enlarged cross-sectional view of the rotational device of FIG. 4 with another embodiment of locking mechanism for the rotational device;

FIG. 5B shows a cross-sectional front view of FIG. 5A;

FIG. 6 shows an enlarged cross-sectional view of the orientation device in FIG. 1 showing rotation of a fixture and a sensor;

FIG. 7 shows an enlarged cross-sectional view of the orientation device in FIG. 1 showing rotation of a device or sensor using one mode of alignment;

FIG. 8 illustrates an enlarged cross-sectional view of the orientation device in FIG. 1 showing rotation of a device or sensor using a different mode of alignment than shown in FIG. 7;

FIG. 9 shows the orientation device of FIG. 8 with the mode of alignment in an alternate position;

FIG. 10 illustrates an enlarged cross-sectional view of the orientation device in FIG. 1 showing rotation of a device or sensor using a different mode of alignment than shown in FIGS. 7 and 8;

FIG. 11 shows an enlarged cross-sectional view of the orientation device in FIG. 1 showing rotation of a device or sensor using a sphere as a joint;

FIG. 12 is an enlarged cross-sectional side view of the orientation device of FIG. 11;

FIG. 13 is an enlarged cross-sectional side view of the orientation device of FIG. 12 having alternative features; and

FIG. 14 is an enlarged cross-sectional view of the orientation device in FIG. 1 having multiple sensors disposed within a single fixture.

### DETAILED DESCRIPTION

The present invention relates to a method and apparatus for orientating a device or sensor, which is also referred to hereinafter as the “implant”, relative to, for example, an external transducer or transmitter, hereinafter also referred to as the “source”. The implant can be configured to perform as, but is not limited to, a receiver or a transmitter depending on the particular embodiment desired. The source can be configured to perform as, but is not limited to, a transmitter, a transducer, a receiver, a power supply, or any combination of the above. In addition, depending on the implementation, the implant is connected by a joint to a fixation structure. The fixation structure includes means to affix the structure to the wall of a bodily lumen.

Orientation adjustments of the sensor device is either manually or automatically achieved. For example, orientation of the implant is attained through mechanical means, like for example, movement by a catheter, or automatically by a force, such as, for example, gravity. Gravitational forces can be utilized by incorporating the implant with a bias weight that makes the implant move in the desired direction. Magnetic forces can be utilized by the attachment of a magnet, for example, to the implant for alignment to the source by application of a magnetic field.

The apparatus of the present invention may be implanted, either temporarily or permanently, within a lumen. The implant may be fixed within a lumen, for example, during an intervention procedure such as implantation of a stent or pace maker, PTCA, or coronary bypass surgery. Depending upon the embodiment, the device may be deployed from a catheter.

The implant may monitor various physiological properties concerning the blood vessel to allow physicians to assess the condition of a patient. In addition, the implant may be used to record or monitor parameters such as, but not limited to, pressure and velocity of flow, biochemical parameters, level of gases and biochemical substances flowing in the fluid contained in the lumen. Depending on the embodiment, the implant may also affect various responses from organs, tissues, or fluids in a patient's body.

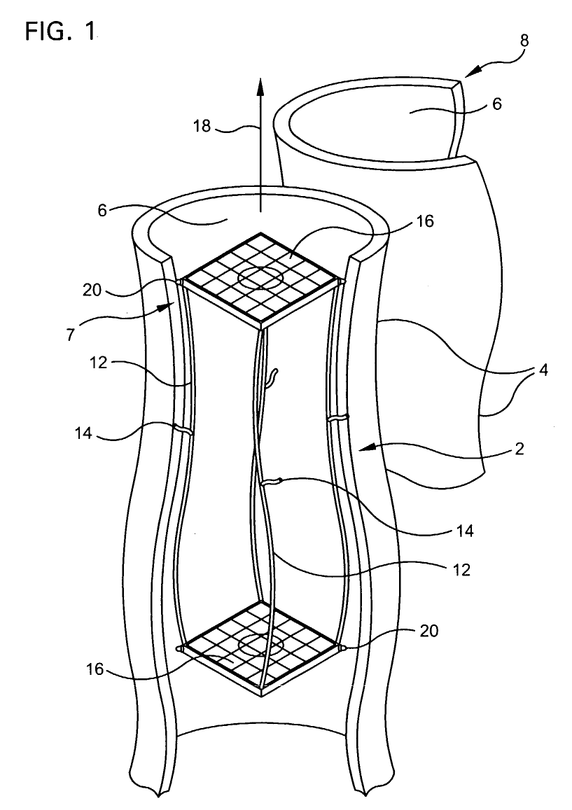

FIG. 1 illustrates one embodiment of an orientation device 2 affixed in a bodily lumen 6. In a preferred embodiment, the orientation device may contain one or more sensors as shown in FIG. 1. Shown in FIG. 1 is a cross-sectional view of an artery 4 having lumen 6 and lumen walls 8.

The orientation device 2 may be inserted in any artery or vessel depending on what properties are desired to be measured. The orientation device includes a fastener 7. The fastener further includes a support fixture 10 and at least one support leg 12. As shown in FIG. 1, multiple support legs may be used to provide further stability of the orientation device 2 in the lumen 6.

As shown in FIG. 1, the orientation device 2 is implanted in a blood vessel or artery 4. The blood vessel 4 has a bodily lumen 6 and a wall 8. Depending on the embodiment, the orientation device 2 is inserted in the bodily lumen 6 through a catheter that has entered the body of a patient. The support leg 12 being compressed in the catheter expands when released from the catheter. Thus, the support legs 12 attach to the wall 8.

To further assist in the attachment process, protrusions 14 are included about the support leg 12. The protrusions can be in the form of, for example but not limited to, a hook, beveled edge, serrated edge, suction cup, medical adhesive or the like. The protrusion 14 may be in the direction of fluid flow 18 within lumen 6 or in the opposite direction or both depending on the desired use of the orientation device. Additionally, the protrusions may be disposed at an end of the support leg, or centrally disposed as shown in FIG. 1. The protrusion may also cover other areas of the support leg to provide additional security in placement of the orientation device 2. The protrusions may be sharp enough to dig into the tissue, however do so with minimum trauma.

The support leg 12 is preferably made of an elastomeric material that is bio-compatible with the human body. In addition, strength of each of the support legs is important to resist the force of the fluid flow 18 within the lumen 6. An example material of this type, although not limited thereto, is nickel titanium (NiTi). The nickel provides the flexibility of the support leg 12, whereas the titanium provides strength and bio-compatibility. The elastic material is strong enough to resists elongation, but resilient to the extent of having spring-like memory when deformed.

The fixture 10 is preferably coupled to an end of the support leg 12. The support leg may connect to another sensor or device as shown in FIG. 1 or hang free to attach to the wall 8. As shown in FIG. 1, the fixture 10 is disposed at approximately the center of the lumen 6, although not limited to such a position. However, it is well known in the art that blood cells concentrate in the center of the lumen. Furthermore, it is well known in the art that the fastest blood flow in a lumen is at its center. Therefore, physiological conditions and biological parameters measured by an implant 16 at the center of the lumen 6 provides a more accurate measurement of the overall conditions and parameters of the lumen 6.

The fixture 10 may be attached to the support leg 12 through a rotational device 20 further described herein. The rotation device 20 allows the fixture to rotate about the support leg 12. Depending on the embodiment, the implant 16 may also rotate in conjunction with or independent of the support fixture 10.

The implant 16 is disposed within the fixture 10. The implant may or may not be permanently attached to the fixture, depending upon the embodiment of the orientation device 2. If the implant 16 is permanently attached, typical methods of bonding may be used such as ultrasonic welding, medical adhesive, mechanical joints or snap fits and the like. The implant is preferably, but is not limited to, a reflective type sensor for reflecting ultrasonic energy from a transducer. Depending on the embodiment, the implant can be an electromagnetic type sensor or any other type of bio-sensor where orientation is beneficial to the measurements obtain through use of a sensor. The implant 16 is not limited to being a sensor, and can be any type of device where orientation of the device is useful.

The implant 16 may be aligned at any angle or position within the lumen 6 to obtain alignment with an external source for measurement of properties of lumen 6. Due to the sensitivity of the sensors that are used for monitoring, which have very thin membranes that are extremely sensitive to mechanical pressure, a coating may be placed on the sensors to protect them from damage and/or destruction during deployment. This optional coating may be made from a material that is soluble in an aqueous solution, and should dissolve immediately or soon after deployment of the sensor. The material used, the thickness of the coating and the hardness of the coating will depend to a large extent on the location, for example, of the sensor, the type of sensor, and a variety of other factors including the physiology involved, the parameters being measured, and the desired speed of deployment.

Examples of a coating is a composition comprising solidified sugar syrup made of approximately equal amounts of glucose and sucrose. The proportions of glucose and sucrose may be varied, however, depending on the application. Another type of a coating is a composition comprising Hydroxy Propyl Methyl Cellulose, Hydroxy Propyl Cellulose and Colloidal Silicone Dioxide, all finely ground and mixed in water, which is used for coating pills and is commercially available as Opadry-Oy-34817 from Colorcon Ltd., Italy. Other materials may also or alternatively be used as a protective coating. The protective coating may be made from any other substance which is hard or thick enough to protect the implant from damage during insertion, dissolves immediately or soon after insertion and is bio-compatible in the intended location of deployment in the body.

If a coating is desired, the implant 16 may be coated by any available method for coating objects including, for example, spraying the coating on the sensor, dipping the implant in a liquid bath, pouring or dripping the coating onto the implant, painting the coating onto the implant, or the like. Additionally, the coating may cover only the membrane of the implant or it may cover a larger portion of the implant or the entire implant.

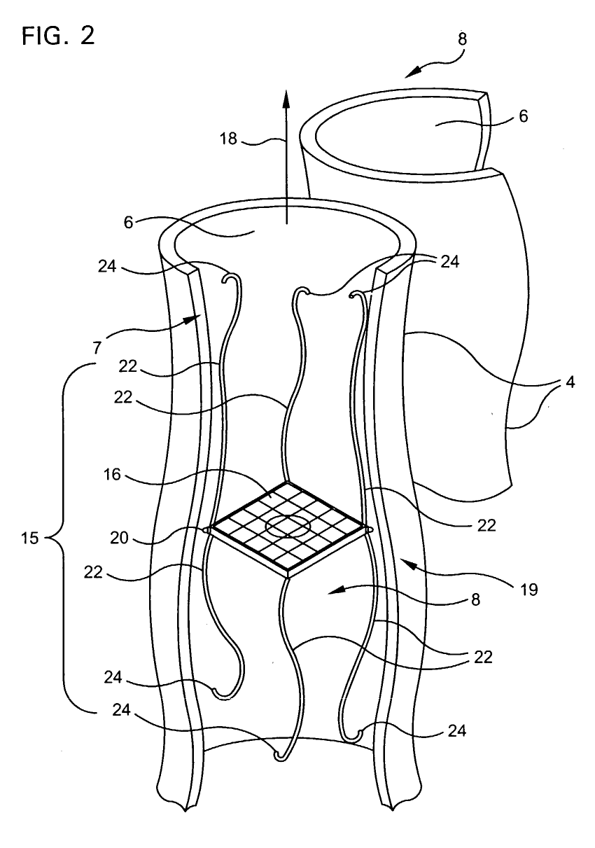

FIG. 2 illustrates another embodiment of the orientation device generally identified by the number 15. Shown is the cross-section view of artery 4 having lumen 6 and lumen walls 8. An orientation device 15 is attached to lumen wall 8.

Orientation device 15 includes a fastener 19. The fastener 19 includes a fixture 10 coupled with support legs 22. In this embodiment, the support legs are coupled to the fixture 10 at one end instead of at both ends as shown in FIG. 1. The multiple support legs may provide additional stability to the orientation device 15.

The support legs 22 further include protrusions 24 similar to the type previously described in FIG. 1. A notable difference in this illustration is that the protrusions in FIG. 1 are both in the same and opposite direction of flow 18. This type of structure may be beneficial where there may be changes in direction of fluid flow 18, or where the vessel is routinely subjected to mechanical forces, either external or internal.

The fixture 10 is also coupled to the support legs 22 through a rotational device 20, although similar to FIG. 1, this structure need not be present depending on the particular embodiment. The implant 16 is further disposed within the fixture 10. In this example, implant 16 is a sensor, similar to the example in FIG. 1. The implant 16 may be aligned in any angle or position, to an alignment with an external source for measurement of properties of the lumen 6.

A patient implanted with the orientation device and may under examination to record measurements through use of an external source. The patient may lie in any position on and an examination table depending on the desired measurement. The position of the patient, in some cases, does not depend on the alignment of the implant and the external source. The implant, for example, may be placed within the chest cavity of the patient. An external source is used to generate energy that is, for example, reflected by the implant. The external source may be a transmitter, a transducer, or both depending on the embodiment.

In one embodiment, the resonance of the energy generated by the source is transmitted back to the source after being reflected by implant. A signal is transmitted, typically through a cable to a processing unit. The processing unit may have a graphical display, a user interface and be supported by a stand. Information of the measurements may be recorded and stored on the processing unit for further investigation.

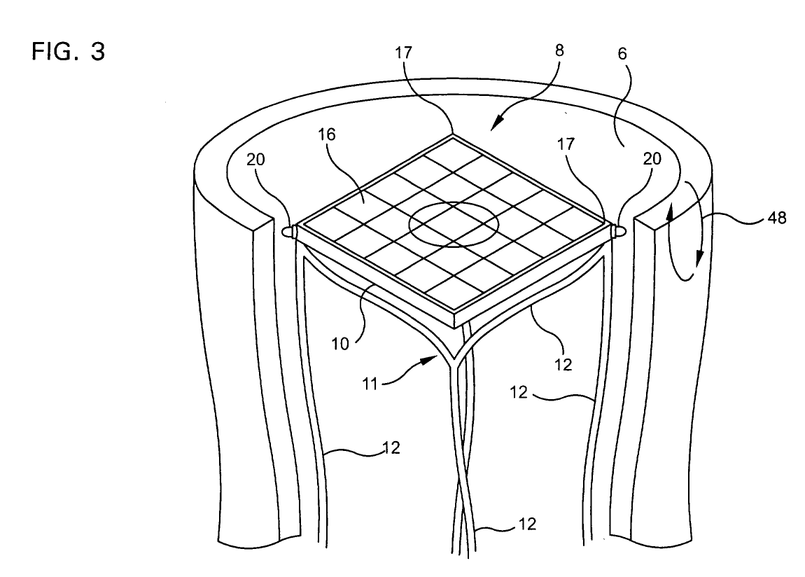

FIG. 3 illustrates an enlarged cross-sectional view of the orientation device showing rotation of the fixture 10 about the support legs 12. This rotation may be used to align the implant 16 with external source 26.

In this illustration, the orientation device is within lumen 6 and support legs 12 are attached to wall 8 as previously shown in FIGS. 1 and 2. The support leg 12 has a split 11 that allows the implant 16 to be rotated about at least two corners 17. The implant 16 may also be rotated from the sides of the implant, and is not limited to being rotated about a fixed position.

In FIG. 3, the support fixture 10 is not fixed and freely rotates, although the invention is not limited to this particular embodiment. The split 11 allows the support leg 12 to couple with other support legs and allow the fixture 10 to freely rotate. Thus, depending on the embodiment, fixture 10 can either be fixed or rotate. In addition, the implant 16 can either be fixed to the support fixture 10 or rotate about fixture 10.

In this example, shown in FIG. 3, implant 16 is fixedly attached to the fixture 10. Thus rotation of the fixture will rotate the implant 16 in the same direction. Rotation of the implant in this figure is designated by arrows marked 48. The implant 16 may be aligned in any position relative to the source by rotating about the rotational device 20. In this embodiment, rotational device 20 is coupled to support leg 12, support fixture 10, and implant 16.

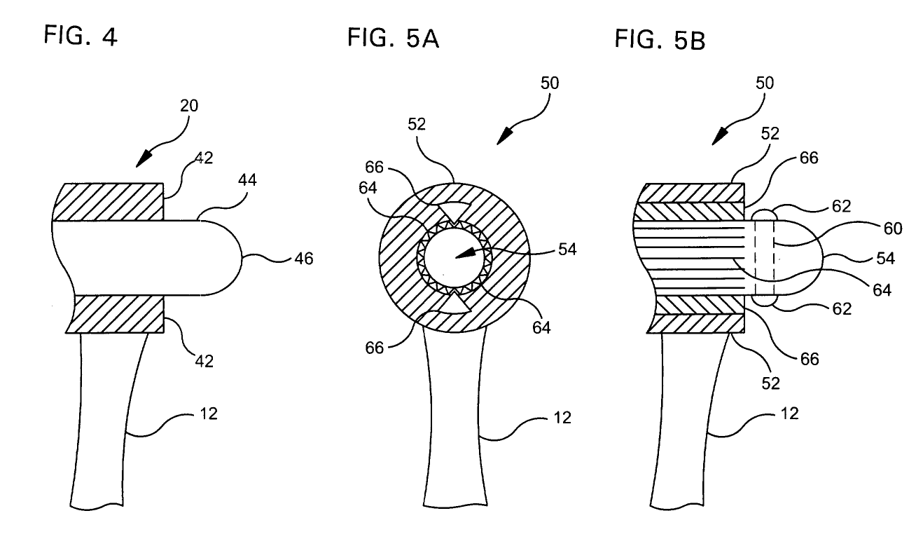

An example of the rotational device 20 is illustrated in FIG. 4. Shown in FIG. 4, is an enlarged cross-sectional view of the rotational device 20 of FIG. 3. This example of the rotational device is just one implementation of how the implant is connected by a joint to the fixation structure, but is not meant to limit the rotation device to this particular embodiment. The implant is able to utilize any other joint connections to connect the implant to the fixation structure. For example, the rotation device can also include, but is not limited to, hinges including living hinges, ball-in-socket joint, pivots, ball bearings, snap fits, any joints, couplings, collars, mechanical fasteners, and any combination thereof.

In the example shown in FIG. 4, the rotational device 20 includes a pin 44 disposed within a tubular structure 42. The tubular structure 42 is attached to the support leg 12. The pin typically has an end 46 which is rounded or has a smooth surface to avoid trauma to the lumen wall 8. The tubular structure may also be attached to the fixture 10 and the implant 16, depending upon the embodiment. The pin 44 is attached to the structure that is desired to be rotated, in this example the implant. However, the implant 16, the fixture 10, or both the implant 16 and fixture 10, can rotate depending on the embodiment. The rotation permits the implant 16 to be aligned in any position the patient may be placed in during the examination procedure.

Once the desired alignment is achieved between the implant 16 and the source 26, the rotational device 20 is preferably locked in position. One example of locking the pin 44 into position is by a frictional fit between the pin 44 and the tubular structure 42. The friction created by the wall of pin 44 and the tubular structure 42 due to dimensional tolerance of each can provide locking strength to hold the pin into the desired position.

In another embodiment, locking of the implant into position is attained by using a gasket disposed about the pin to interact with the wall of the tubular structure 42. Again, friction is the mechanism used to hold the pin 44 in position. However, by using a gasket, various coefficients of friction may be obtained without compromising the material integrity of the pin 44.

FIGS. 5A and 5B further illustrate another locking mechanism that can be utilized in the orientation device to lock the implant 16 into position once alignment is achieved. Shown is an enlarged cross-sectional view of a rotational device 50.

Rotational device 50 includes a pin 56 disposed within a tubular structure 52. The tubular structure 52 is attached to support leg 12. The pin 56 is attached to the implant, the fixture, or both depending on the desired rotation. To prevent the pin 54 from sliding out of tubular structure 52, a security pin 60 may be installed at end 56. Similar to FIG. 4, end 56 is rounded or smoothed to minimize trauma to the lumen wall 8. Security pin 60 may further include ends 62 that are also shaped to minimize trauma. The ends 62 further prevent the pin 54 from withdrawing into the tubular structure 52.

In the example shown in FIG. 5A, the pin 54 further includes serrations 64. These serrations can circumferentially encompass the pin to form a gear shaped configuration, yet need not do so for the locking feature to operate. For example, the serrations may cover only a portion of the pin desired to be rotated. In this example, the serration 64 is designed to rest on locking tooth 66. As shown in FIG. 5B, which is a front view of the rotational device 50 of FIG. 5A, the rotational device may have one or more than one locking tooth 66 to engage with the serrations 64 of pin 54. Once the desired alignment between the implant and the external source is achieved, the serrations 64 and the locking tooth 66 resist any further movement of the pin and the alignment is locked into position. As previously, stated the implant, fixture, or both structures may be rotated into the desired alignment position.

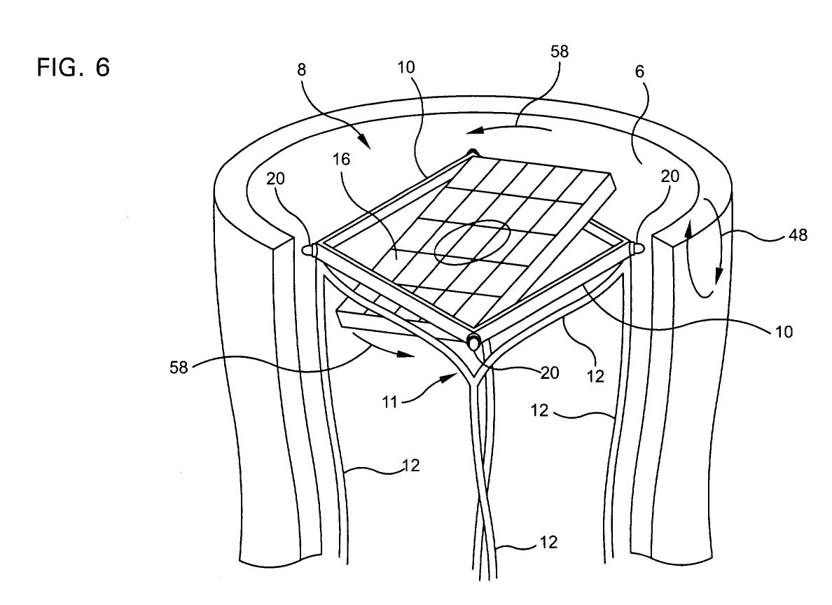

Turning to FIG. 6, shown is an enlarged cross-sectional view of the orientation device. In this embodiment, both the implant and the fixture rotate within the lumen 6. The implant and fixture may both rotate independently or dependently upon each other, depending on the embodiment.

The orientation device shown in FIG. 6, allows both the implant and the fixture to rotate independent of each other so that any position of the implant 16 is obtainable. The orientation device, in this example, includes a secondary fixture 70 that holds additional rotational devices 20 to allow the implant to freely rotate. As shown in FIG. 6, rotation device 20 is attached to fixture 10 allowing the frame to rotate in a direction designated by arrows marked 48. It is within the scope of this invention that the rotational devices allow rotation both in the clockwise and counter-clockwise positions. The fixture 10, depending on the embodiment, can also contain additional rotational devices 20 that are connected to the implant 16. In the example shown in FIG. 6, the implant utilizes a secondary frame 70 that is connected to the rotational devices. The implant can freely rotate in a direction designated by an arrows 58 independent of the rotation of the fixture 10. This flexibility allows the implant 16 to be positioned in any direction or angle.

The following remaining figures are described with the fixture 10 being fixed and the implant 16 rotating. These embodiments are given only as example of the principles of the invention and are not intended to limit these embodiments to a fixed fixture. As previously illustrated, it is within the scope of the invention for the implant, support fixture, or both to rotate for obtaining alignment of the implant 16 with the source 26. With any of the following embodiments, the locking mechanisms previously described may or may not be incorporated to lock the position of the sensor.

The following examples illustrate various modes or ways orientation can be achieved for alignment between the implant and the source. These description are in no way meant to limit the invention to the three embodiments described below and are merely exemplary of the principles of the invention. It is within the scope of this invention that the three modes of alignment described below may be used alone or in any combination with each other.

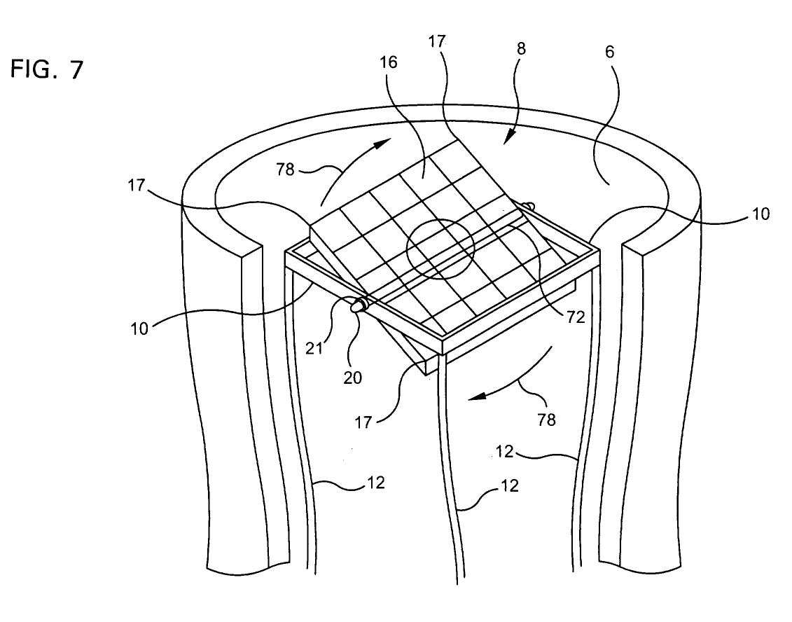

Turning now to FIG. 7, shown is an enlarged cross-sectional view of one mode of alignment of the sensor. Explained here and in the following embodiments are modes of alignment that force the implant 16 to rotate in alignment with the external source 26.

The mode of alignment shown in FIG. 7 is a physical manipulation mode. Shown is the implant 16 freely rotating about fixed fixture 10 in a direction marked by an arrow designated as 78. Again the rotation of the implant may be in the reverse direction as indicated by the arrow 78. A pin 72 may be placed through the implant 16 and into the rotational device 20 to allow the implant to rotate. Illustrated in this example, the pin 72 is mounted on a side 21 of implant 16 instead of corners 17 previously mentioned. This example is not meant to limit the previous embodiments to rotating on the corners 17 and is within the scope of the invention that the previous embodiments may also rotate about side 21 of implant 16. Alternatively, the pin 72 may be integrally formed as a protrusion of the implant and need not be put through the entire implant structure. As previously stated, there may be mechanisms incorporated in the rotational device 20 to lock the pin into position once the desired alignment is achieved.

For the implant to rotate in the embodiment shown in FIG. 7, a physical force must initiate the rotation of implant 16. Such physical force may be obtained by using the end of a catheter to push or rotate the implant in the desired position. Measurements with the external source may be taken to determine when the strongest signal is received and what position the implant is in to receive such signal. At that point, the implant may be locked into place using methods and mechanisms previously described.

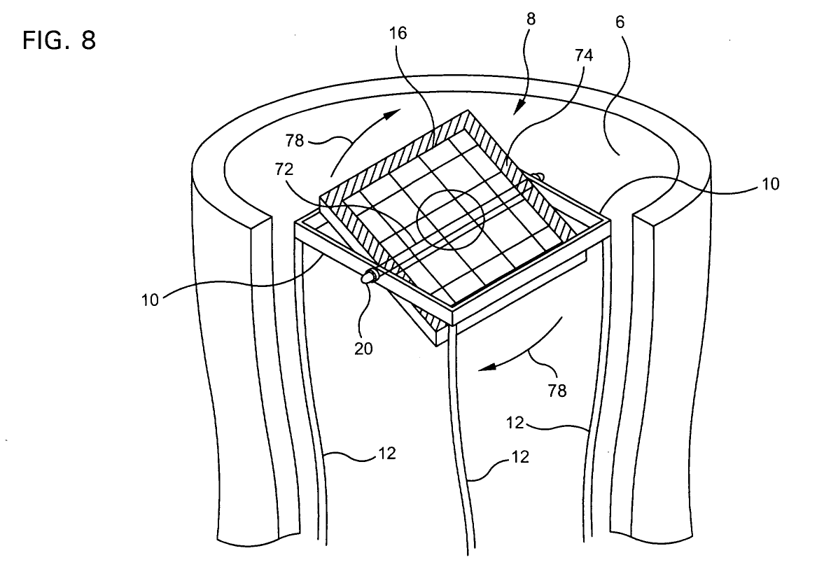

FIG. 8 illustrates a second mode of alignment. Shown is a cross-section view inside lumen 6 of the orientation device utilizing a magnetic force to rotate and align the implant 16 with the external source 26.

The implant 16 in FIG. 8 includes a face plate 74 that may consist of a magnetic material. The magnetic material of face plate 74 is attracted to the metal of source 26 and will automatically align the implant with the source when the source is placed near the patient that has the orientation device implanted. Alternatively, the face plate may be composed of a metallic material and the source composed of a magnetic material, such that when the source is placed over the orientation device the implant will align with the source. In addition, both the implant and the source may be composed of magnetic material so that the source repels the implant in the opposite direction. This embodiment may be desirable if more than one source is needed for the desired measurements.

The magnetic material of face plate 74 requires no external energy to operate, and therefore requires no internal power source. However, if the desired magnetism is to be made with an internal power source, a magnetic field may be created only when needed for alignment purposes. For example, a wound coil can produce a magnetic field when voltage is run through it. The magnetic field may then be activated only when alignment is required by supplying the power through the coil at the desired time.

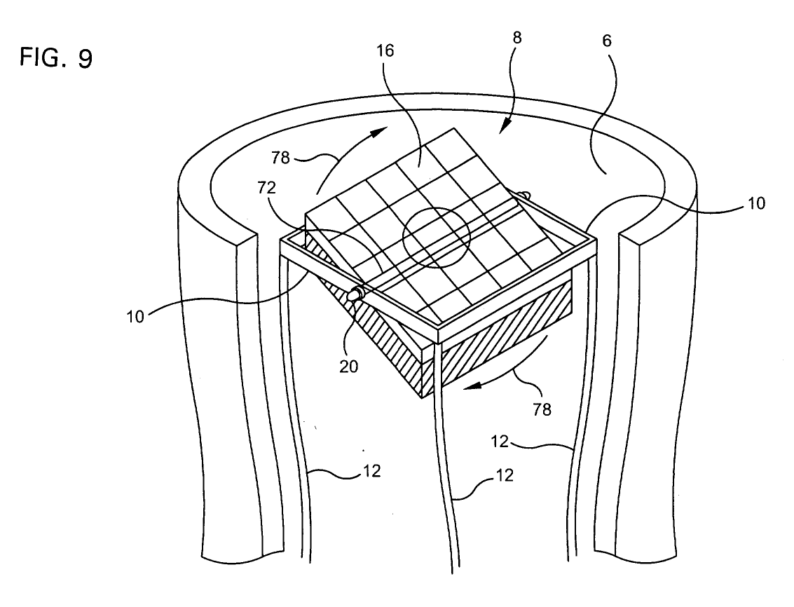

FIG. 9 illustrates a similar mode of alignment as FIG. 8 with the face plate 74 being replaced with bottom 80. Shown is the cross-section view of FIG. 8 inside lumen 6 of the orientation device utilizing magnetic force to rotate and align the implant 16 with the source 26.

In certain circumstances, the existence of face place 74 may interfere with the implant's response to the transmission of source. In these circumstances, it may be beneficial to position the magnetic material or attractive metallic material on the back portion of implant 16 as shown in FIG. 9. The back portion of implant 16 may be constructed so that the attractive magnetic forces are in alignment with the direction required for the implant to respond to the source.

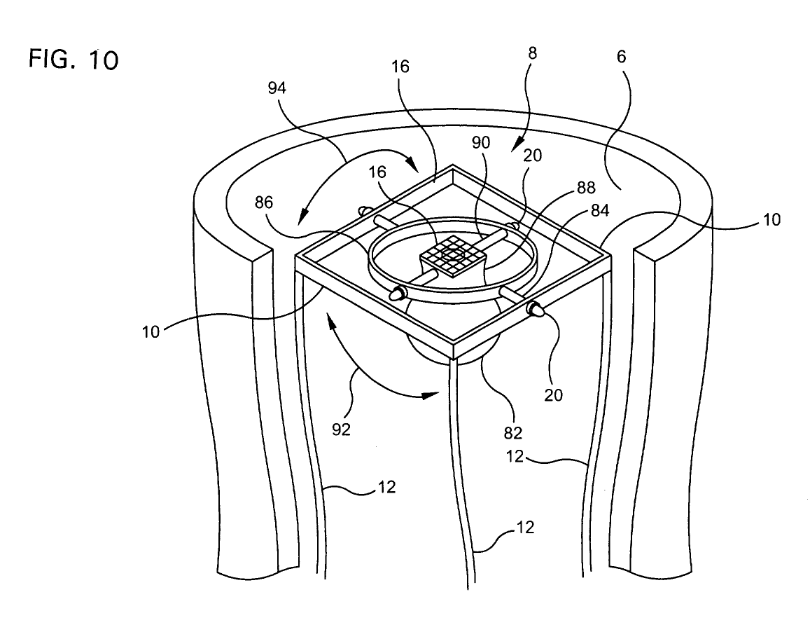

FIG. 10 illustrates a third mode of alignment utilizing gravitational force. Shown is an enlarged cross-sectional view of lumen 6 with the orientation device utilizing a weighted implant 88.

The weighted implant 88 is constructed so that its center of gravity provides motion to the implant when the patient changes position during the examination procedure. A bottom 82 of weighed implant 88 is designed so that the center of gravity of weighted implant 88 is below implant 16. Thus, as the patient changes positions the bottom 82 will move relative to gravity and therefore move the implant 16. The implant 16 is mounted on top of the weighted implant 88. A rotational pin 90 is connected to the weighted implant for providing rotation in the directions generally indicated by arrows 92.

The pin 90 is further coupled to rotational device 20, which is mounted on a rotational ring 86. Rotational ring 86 provides structure for the rotational device 20 that allows the weighed implant to rotate in the directions of arrow 92. The rotational ring 86 need not be a ring and is given as only an example. Rotational ring may be any shape or structure that allows the weighed implant to rotate and provides a structure to hold the rotational device 20.

Rotational ring 86 is further coupled to pin 84 and additional rotational devices 20 that are coupled to the fixture 10. Again, in this example, the fixture does not rotate, however, as previously described in detail, the fixture may rotate as well. Pin 84 allows the implant to rotate in directions marked by arrow 94.

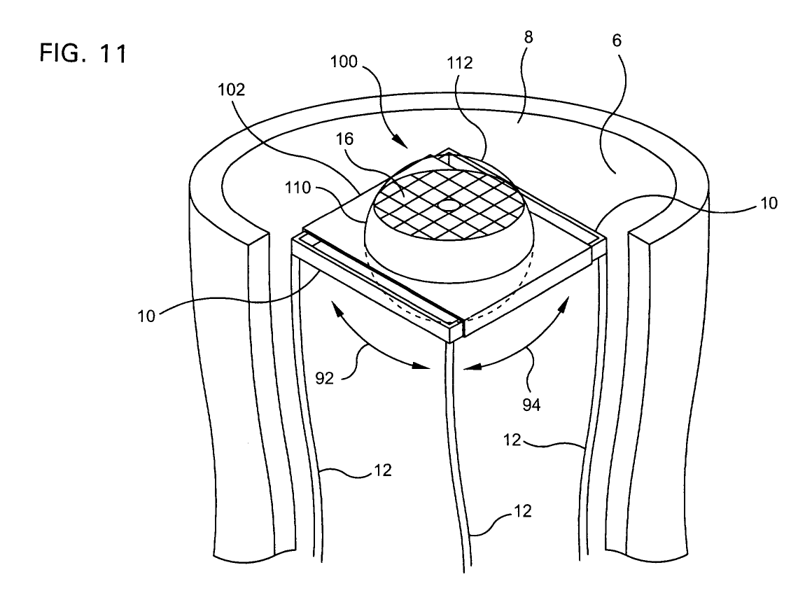

An alternative embodiment of the orientation device that can utilize any of the alignment modes previously described is shown in FIG. 11. Shown is an enlarged cross-sectional view of a orientation ball device 100 inside lumen 6 that utilizes a different means of rotation other than that provided by rotational device 20.

The orientation ball device includes a sphere 110 holding the implant 16. Depending on the embodiment, the implant can be further protected with a implant cover 112 that is disposed over the implant and is attached to sphere 110. The cover 112 is preferably made of a material that does not interfere with the reflective properties of the implant. The orientation device 100 is supported by a ball support 102 that is coupled to the fixture 10.

The rotation device 20 is removed from the orientation device in this embodiment and replaced with a sphere 110. The sphere acts similarly to the ball found at the bottom of a computer mouse in that the sphere 110 can rotate in any direction. The orientation ball device is free to rotate in any position or direction about ball support 102. Locking mechanisms previously described can be incorporated into ball support 102 to lock the implant 16 into position. For example, serrations(not shown in this illustration) on the sphere 110 can lock into locking teeth incorporated in the ball support 102.

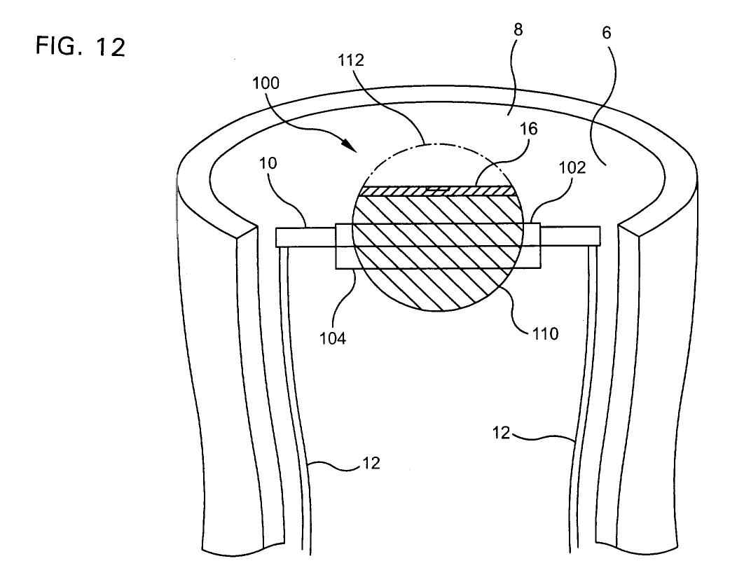

FIG. 12 is a side view of the ball orientation device shown in FIG. 11. The ball support 102 can be supplemented with a lower ball support 104 as shown in FIG. 12. Supports 102 and 104 prevent the sphere 110 from dropping out of fixture 10. The supports may or may not be made of the same material as the fixture depending on the embodiment. Preferably the supports 102 and 104 and sphere 110 are made of material that allow a low coefficient of friction to allow the sphere 110 and thus the implant 16 to freely rotate at any angle or position.

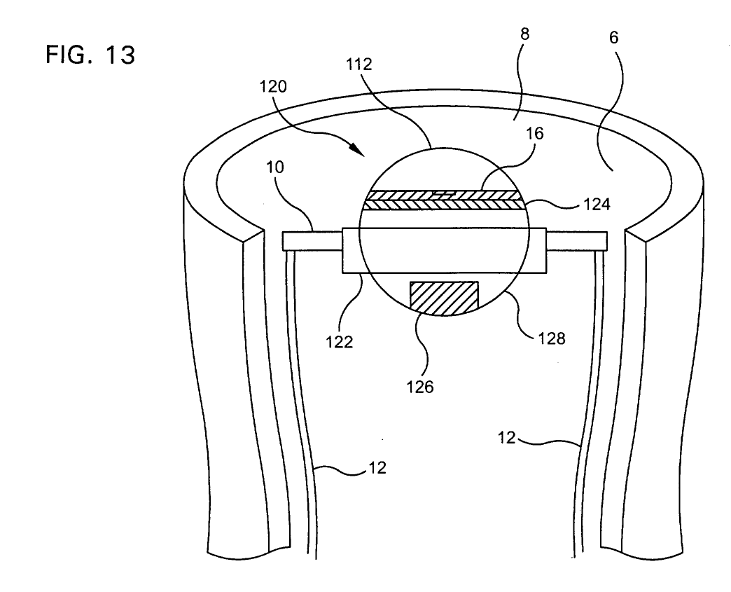

FIG. 13 illustrates a ball orientation device 120 disposed in lumen 6. Shown is an enlarged side cross-sectional view of the orientation device affixed to lumen wall 8.

The orientation device 120 is without the cover 112, but may have one attached depending upon the embodiment. Implant 16 is positioned on top of a sphere 128. The sphere 128 has sphere supports 122 which function similarly to supports 102 and 104. In this embodiment, there is a unitary support structure that prevent the sphere 128 from dropping out of the fixture 10.

The sphere 128 further includes a plurality of placement slots 124 and 126. Slots 124 and 126 can be utilized to hold additional implants and/or magnetic related material as previously described to force alignment of the implant with the external source. The locking mechanisms previously described may also be incorporated into the orientation device 120.

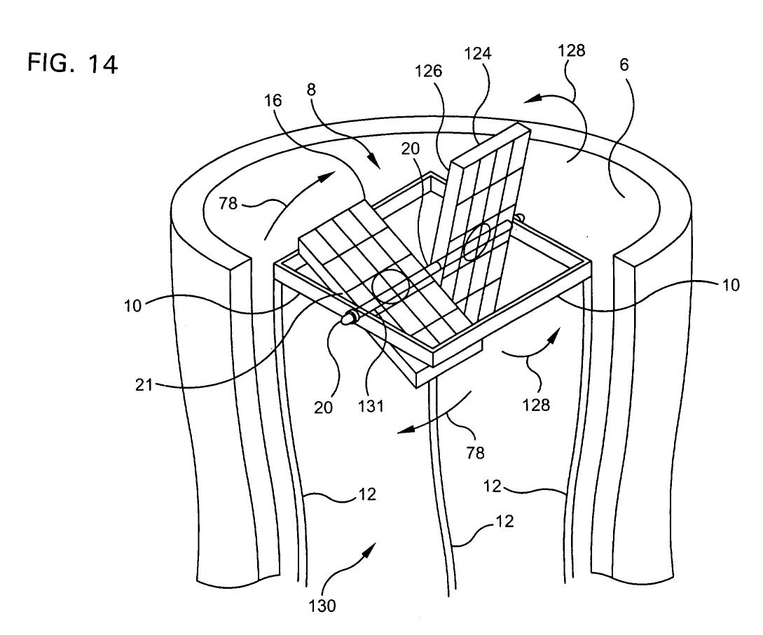

FIG. 14 illustrates the use of multiple implants or sensors utilized in the orientation device 130. Again for illustration purposes only, this example demonstrates the movement of the implants about a fixed fixture. However, as previously stated, the implant, fixture or both may rotate.

Orientation device 130 is disposed in lumen 6 and affixed to lumen wall 8. The implant 16 is allowed to rotate using modes of alignment previously described. Motion in the direction of arrows marked 78 or in the opposite direction are possible through the rotation device 20 attached to side 21 of the implant. Again, the implant is not limited to connection at the side of the implant and may be attached to the rotational device at any place along the periphery of the implants.

A secondary implant 124 is also included in the orientation device 130. The secondary implant may or may not rotate independently of implant 16, depending upon the embodiment. In the example illustrated, implant 124 is attached to another rotational device 20 on the fixture 10. This secondary rotational device 20 allows the implant 124 to rotate independently of implant 120 as indicated by a direction marked by arrows 128. If it is desirable for the implants to rotate dependent of each other, the rotational device 20 can utilize a common pin 131 that may be attached to sides 126 and sides 21 and run through the implants.

The operation or method of use of the orientation device is now described. The orientation device is placed in the body lumen and the implant is aligned with the external source. The orientation device placement may or may not include deployment using a catheter. If the catheter is used, the support legs are compressed within the catheter prior to deployment. Upon release of the orientation device from the catheter, the support legs expand and attach to the wall of the lumen.

The position of the implant relative to the source, depending upon the embodiment, may be fixed by manually rotating implant, the fixture, or both. A catheter may be used such as, for example, the catheter used to deploy the orientation device.

In addition, the position of the implant may be adjusted by using magnetic force. In this situation, the implant, the source, or both, may possess magnetic material. For example, a patient lies on an examination table. In the case of the implant or the source containing the magnetic material, the source is placed about the patient to attract the implant in alignment with source.

Thus, regardless of the position of the patient, the source is in alignment with the implant due to magnetic force and attraction. When both the implant and the source contain the magnetic material, repulsion of opposing magnetic forces assist in alignment of the implant.

Similarly, a chemical reaction may cause the implant to rotate or move into alignment with the source. For example, a chemical reaction initiated by the energy of the source may create a gradient that may force the implant into alignment with the source.

In addition, gravitational forces may also assist in the alignment of the implant with the source. For example, in the case of a weighted implant, the center of gravity about the implant will force the implant in alignment with source based upon a patient's position.

It should be understood that the above description is only representative of illustrative examples of embodiments and implementations. For the reader's convenience, the above description has focused on a representative sample of possible embodiments, a sample that teaches the principles of the present invention.

Other embodiments may result from a different combination of portions of different embodiments. The description has not attempted to exhaustively enumerate all possible variations. The alternate embodiments may not have been presented for a specific portion of the invention, and may result from a different combination of described portions, or that other undescribed alternate embodiments may be available for a portion, is not to be considered a disclaimer of those alternate embodiments. It is appreciated that many of those undescribed embodiments are within the literal scope of the following claims, and others are equivalent.

What is claimed is:

1. A method of orientating a device or a sensor in a bodily lumen, comprising:
placing in the bodily lumen a fastener of the type comprising at least one support leg, an implant and a fixture coupled to the support leg for holding the implant; and
aligning the implant with a source external to the bodily lumen.

2. The method of claim 1, wherein the implant is temporarily fixed in the bodily lumen.

3. The method of claim 1, wherein the implant is a sensor or a device to affect or monitor physiological conditions within the bodily lumen.

4. The method of claim 1, wherein the fastener placement is done using a catheter.

5. The method of claim 4, wherein the alignment further includes rotating the fixture.

6. The method of claim 5, wherein the rotation further includes setting the position of the fixture with the catheter.

7. The method of claim 5, wherein the rotation further includes setting the position of the fixture by using magnetic force.

8. The method of claim 5, wherein the rotation further includes setting the position of the fixture by positioning a patient having the implant in various positions.

9. The method of claim 5, wherein the rotation further includes rotating the implant independent of the fixture.

10. The method of claim 1, wherein the alignment further includes rotating the implant.

11. The method of claim 10, wherein the implant rotation further includes rotating the fixture independent of the sensor.

12. The method of claim 1, wherein the alignment further includes moving the implant by a physical force.

13. The method of claim 1, wherein the alignment further includes moving the implant by a magnetic force.

14. The method of claim 1, wherein the alignment further includes moving the implant by gravitational force.

15. The method of claim 1, wherein the alignment further includes moving the implant by using a chemical reaction.

16. The method of claim 1, wherein the alignment further includes moving the source about a patient to position the implant.

17. The method of claim 1, wherein the source is a transmitter, a transducer, a power supply, a receiver, or any combination thereof.

18. A method of orientating a device or a sensor in a bodily lumen, comprising:
inserting in the bodily lumen a fastener of the type comprising at least one support leg having a first end and a second end, an implant, a fixture coupled to the first end of support leg for holding the sensor, and at least one protrusion located on the support leg;
attaching the fastener on a wall of the bodily lumen by the protrusion; and
aligning the implant with a source external to the bodily lumen of a patient.

19. The method of claim 18, wherein the fastener insertion further includes inserting a catheter into the bodily lumen.

20. The method of claim 18, wherein the fastener inserting further includes inserting a stent into the bodily lumen.

21. The method of claim 18, wherein the fastener attachment further includes releasing the fastener from a catheter.

22. The method of claim 18, further includes moving the source about the patient having the implant to receive information from the implant.

23. The method of claim 18, further includes moving the source about the patient having the implant to change physiological conditions within the bodily lumen.

24. The method of claim 18, wherein the implant alignment further includes moving the implant with a catheter.

25. The method of claim 18, wherein the implant alignment further includes moving the source about the patient having the implant in order to move the implant into alignment with the source.

26. The method of claim 25, wherein the implant movement is caused by magnetic force.

27. The method of claim 18, wherein the implant alignment further includes changing the position of the patient to move the implant into alignment with the source.

28. The method of claim 27, wherein the implant movement is caused by gravitational force.

29. The method of claim 27, further includes receiving information from the implant.

30. A method of orientating a sensor or device in a bodily lumen, comprising:
inserting in the bodily lumen a catheter having disposed therein a compressed fastener of the type comprising at least one support leg with a first end and a second end, an implant, a fixture coupled to the first end of support leg for holding the sensor, and at least one protrusion located on the support leg;
releasing the fastener from the catheter at a desired location in the bodily lumen of a patient;
affixing the fastener by allowing the fastener to expand in the bodily lumen so the fastener attaches to a wall of the bodily lumen by the protrusion; and
aligning the implant with a source external to the bodily lumen.

31. The method of claim 30, wherein the alignment includes rotating the implant.

32. The method of claim 30, wherein the alignment includes rotating the fixture.

33. The method of claim 30, wherein the alignment further includes positioning the implant by moving the catheter.

34. The method of claim 30, wherein the alignment includes moving the implant by moving the transducer.

35. The method of claim 34, wherein the implant movement is by magnetic force.

36. The method of claim 30, wherein the alignment includes moving the implant by positioning the patient.

37. The method of claim 36, wherein the implant movement is by gravitational force.

38. The method of claim 30, further including taking a measurement from the implant.

39. The method of claim 30, wherein the alignment further includes locking the implant into a desired position relative to the source.

40. An orientation device for placement in a bodily lumen, comprising:
a fastener having at least one support leg;
a fixture coupled to the support leg, holding an implant; and
means for aligning the implant with a source external to the bodily lumen.

41. The device of claim 40, wherein the support leg includes at least one protrusion.

42. The device of claim 41, wherein the protrusion is positioned at one end of the support leg.

43. The device of claim 41, wherein the protrusion is positioned about the middle of the support leg.

44. The device of claim 40, wherein the alignment means includes mechanical means, gravitational means, magnet means, or any combination thereof.

45. The device of claim 44, wherein the magnet means is disposed on the implant.

46. The device of claim 44, wherein the magnet means is disposed on the source.

47. The device of claim 44, wherein the mechanical means is attached to the support leg for moving the fixture.

48. The device of claim 47, wherein the mechanical means further includes a secondary mechanical means attached to the fixture for moving the implant.

49. The device of claim 44, wherein the fixture is stationary and the mechanical means is attached to the fixture to move the implant.

50. The device of claim 40, wherein the implant is a sensor or a device to affect or monitor physiological conditions within the bodily lumen; and the source is a transmitter, a transducer, a power supply, a receiver, or any combination thereof.

51. The device of claim 44, wherein the mechanical means further includes a pin.

52. The device of claim 51, wherein the pin couples the implant and the fixture.

53. The device of claim 51, wherein the pin couples the fixture and the support legs.

54. The device of claim 41, further including a locking means.

55. The device of claim 54, wherein the locking means further includes serrated teeth disposed about the pin.

56. The device of claim 54, wherein the locking means further includes a frictional fit receiving tube to receive the pin.

57. The device of claim 56, further including a gasket disposed in the tube.

58. The device of claim 54, wherein the locking means further includes a snap fit.

59. An orientation device for placement in a bodily lumen, comprising:
a fastener having at least one support leg having a first end and a second end, the support leg further having at least one protrusion for attaching the fastener on a wall of the bodily lumen;
a fixture coupled to the first end of support leg, holding an implant; and
means for aligning the implant with a source external to the bodily lumen.

60. The device of claim 59, wherein the alignment means is located on the support legs for rotating the fixture relative to the support legs.

61. The device of claim 59, wherein the alignment means is located on the fixture for rotating the implant relative to the fixture.

62. The device of claim 59, wherein the alignment means is located on both the support legs and the fixture.

63. The device of claim 59, wherein the protrusion is oriented in a direction similar to fluid flow in the bodily lumen.

64. The device of claim 59, wherein the protrusion is oriented in both a direction similar and a direction opposite to fluid flow in the bodily lumen.

65. The device of claim 59, further including a second fixture couple to the second end of the support leg.

66. The device of claim 59, further including at least one sphere disposed within the fixture for holding the implant.

67. The device of claim 66, wherein the alignment means further includes at least one sphere support.

68. The device of claim 66, wherein the sphere includes a plurality of implants.

69. The device of claim 59, wherein the alignment means includes a weighed implant.

70. The device of claim 59, wherein the alignment means includes a magnetic implant.

71. The device of claim 59, wherein the alignment means includes a pin.

72. The device of claim 71, wherein the pin is attached to the support leg.

73. The device of claim 71, wherein the pin is attached to the implant.

74. The device of claim 71, wherein the pin is attached to the implant and the support leg.

75. The device of claim 71, wherein the pin further includes a locking means.

76. An orientation device for placement in a bodily lumen, comprising:
a fastener having at least one support leg having a first end and a second end, the support leg further having at least one protrusion for attaching the fastener on a wall of the bodily lumen;
a fixture coupled to the first end of support leg;
a primary implant disposed in the fixture for monitoring properties of the bodily lumen;
means for aligning the implant with a source external to the bodily lumen; and
a catheter for inserting the fastener into the bodily lumen.

77. The device of claim 76, wherein alignment means includes a joint attached to the implant and the catheter physically rotates the implant about the joint.

78. The device of claim 77, wherein the joint is a pin, a ball-in-socket joint, a hinge, a collar, a coupling, a snap-fit, a pivot, a ball bearing, or any combination thereof.

79. The device of claim 76, wherein the alignment means includes a pin attached to the support leg and the catheter physically rotates the fixture about the pin.

80. The device of claim 76, wherein the support legs and the fixture is made of an elastomeric material.

81. The device of claim 80, wherein the elastomeric material comprises nickel titanium.

82. The device of claim 76, further including a secondary fixture having a secondary implant coupled to the second end of the support leg.

83. The device of claim 76, wherein the implant further includes a protective coating.

84. The device of claim 76, wherein the implant is a reflective sensor.

85. The device of claim 84, wherein the implant further includes a material for reflecting ultra sound waves transmitted by the source.

86. The device of claim 76, further including a secondary implant disposed in the fixture with the primary sensor.

87. The device of claim 86, wherein the secondary implant is positioned at a different angle as the primary implant for providing information about the bodily lumen.

88. The device of claim 76, wherein the source is a transducer or transmitter.
